(19) United States
(12) Patent Application Publication
(10) Pub. No.: US 2023/0166917 A1
(43) Pub. Date: Jun. 1, 2023

(54) LID TRANSPORT CONVEYOR AND A TRANSPORT AND DECONTAMINATION MACHINE

(71) Applicant: SYNERLINK, PUISEUX PONTOISE (FR)

(72) Inventors: Patrick MASSOT, JOUE L'ABBE (FR); Stéphane GROGNET, EAUBONNE (FR); Laurent DANDY, CONFLANS SAINTE HONORINE (FR); Nathanael DESBLEDS, CHAMPAGNE SUR OISE (FR)

(73) Assignee: SYNERLINK, PUISEUX PONTOISE (FR)

(21) Appl. No.: 18/071,851

(22) Filed: Nov. 30, 2022

(30) Foreign Application Priority Data
Nov. 30, 2021 (FR) 2112743

(51) Int. Cl. B65G 15/58; B65G 15/48; B65G 47/06; A61L 2/26; A61L 2/10; A61L 2/18

(52) U.S. Cl. CPC B65G 15/58; B65G 15/48; B65G 47/06; A61L 2/26; A61L 2/10; A61L 2/186

(57) ABSTRACT

A lid transport conveyor including a frame, a first pulley and a second pulley that are carried by the frame, a device for driving the first pulley in rotation, an endless conveyor belt mounted between the first pulley and the second pulley, a slide profile carried by the frame, the slide profile including a suction groove whose open side directly faces a main face of the conveyor belt, a pump capable of drawing air through the suction groove, conveyor belt including a through-orifice positioned in line with the suction groove during movement of the conveyor belt; a lid being held by suction in contact with a face of the conveyor belt that is opposite to the main face, when the orifice is directly opposite the suction groove.

### TECHNICAL FIELD

This invention relates to a lid transport conveyor and to a transport and decontamination machine comprising such a conveyor.

### BACKGROUND

There are known lid transport conveyors in which the lids are positioned and held on plates equipped with pins. These systems have the disadvantage of being dedicated to a single lid format and must be changed each time the lid format is changed.

### SUMMARY

A first object of this invention is to provide a conveyor capable of transporting lids of different formats without changing tools in the conveying portion.

A second object of this invention is to provide a conveyor which does not require the use of plates equipped with pins to hold the lids, which are dedicated to the format of the lid.

A third object of this invention is to provide a machine which allows decontaminating the lids while transporting them.

A fourth object of this invention is to provide a more hygienic conveyor.

A fifth object is to provide a more ergonomic conveyor.

One object of this invention is a lid transport conveyor comprising:
a frame,
at least a first pulley and a second pulley which are carried by the frame,
a rotation-driving device capable of rotating at least the first pulley,
at least one endless conveyor belt mounted between the first pulley and the second pulley, the conveyor belt extending in a longitudinal direction, the conveyor belt being driven to move by the first pulley,
at least one slide profile carried by the frame, the slide profile comprising a first suction groove extending in the longitudinal direction, the open side of the first suction groove directly facing a main face of the conveyor belt,
at least one pump capable of drawing air through said first suction groove, said conveyor belt comprising at least one through-orifice positioned, at least at times, in line with the first suction groove, during movement of the conveyor belt; at least one lid being held by suction in contact with a face of the conveyor belt that is opposite to the main face, when the orifice is directly opposite the first suction groove,
   characterized in that the slide profile comprises at least one second suction groove aligned with and in the extension of the first suction groove, the open side of the second suction groove directly facing a main face of the conveyor belt, said at least one pump being capable of drawing air through said second suction groove.

Advantageously, the suction may be used or stopped in the first suction groove or second suction groove independently, to facilitate lid transport along the first suction groove and lid removal along the second suction groove.

According to one embodiment, the same pump is used to draw air through the first suction groove and through the second suction groove. According to another embodiment, two different pumps are used to draw air through the first suction groove and through the second suction groove.

The features set forth in the following paragraphs may optionally be implemented. They may be implemented independently of each other or in combination with each other:
the at least one conveyor belt comprises at least one set of several orifices grouped and aligned along the longitudinal direction, and the center-to-center distance between the orifices furthest apart within a set of orifices is greater than the thickness of the wall between the first suction groove and the second suction groove.
the at least one conveyor belt comprises at least one oblong orifice extending in the longitudinal direction, the oblong orifice having a first end and an opposite second end along the longitudinal direction; the slide profile comprising a wall between the first suction groove and the second suction groove, and the center-to-center distance between the first and second ends of the oblong orifice is greater than the thickness of the wall between the first suction groove and the second suction groove.

The slide profile comprises at least one additional first suction groove parallel to the first suction groove, and the conveyor belt comprises at least one additional orifice, the at least one additional orifice of the conveyor belt being arranged, at least at times, in line with said first additional suction groove, during movement of the conveyor belt.

Advantageously, the use of two suction grooves makes it possible to improve the fixing of the lids against the conveyor belt.

The conveyor comprises several slide profiles and several conveyor belts extending in the longitudinal direction, the slide profiles being parallel to each other and arranged next to each other in a transverse direction, the conveyor belts being parallel to each other and arranged next to each other in a transverse direction. The conveyor belt is made of stainless steel.

The at least one slide profile is arranged between an upper horizontal portion of the at least one conveyor belt and a lower horizontal portion of the at least one conveyor belt.

The conveyor further comprises a lid storage magazine among a first lid storage magazine and a second lid storage magazine, the first lid storage magazine and the second lid storage magazine being suitable for mounting on the frame in a removable manner, the first lid storage magazine including a plate comprising dispensing openings having a first diameter, the second lid storage magazine including a plate comprising dispensing openings having a second diameter that is different from the first diameter.

The conveyor comprises N slide profiles and N conveyor belts, the number N being a positive integer greater than 1, and the plate of the first lid storage magazine comprises N dispensing openings, the plate of the second lid storage magazine comprising N/2 dispensing openings.

The conveyor comprises a lid destacking device capable of transferring the lids from the lid storage magazine to the at least one conveyor belt, the destacking device comprising:
at least two gripping members rotatably mounted relative to a vertical axis arranged between the two gripping members,
a rotation-driving device capable of rotating the gripping members about the vertical axis;
the gripping members being mounted to be vertically movable in translation,
a translation-driving device capable of driving the gripping members in translation between a high position for picking up/removing lids and a low position for transferring lids.

The lid storage magazine is fixed to the frame in the extension of the slide profiles along the longitudinal direction, and the destacking device is positioned below the lid storage magazine and below one end of the slide profiles, the vertical axis being arranged between one end of the slide profiles and the lid storage magazine.

Advantageously, the lid storage magazine is located about 1 meter from the ground so that it is easier to load.

The slide profile comprises at least one unloading suction groove, aligned with the first suction groove, the open side of the unloading suction groove directly facing a main face of the conveyor belt, said at least one pump being capable of drawing air through said unloading suction groove; the conveyor comprising a removal device for removing at least one lid arranged in line with an unloading suction groove, and a dispensing unit capable of controlling the at least one pump to draw air into the at least one first suction groove and into the unloading suction groove during a lid transport and/or lid loading phase, and to draw air only into the unloading suction groove during a phase of unloading at least one lid by the removal device.

Advantageously, the existence of at least two suction grooves aligned with each other, namely the first suction groove and the unloading suction groove, makes it possible to unload the lids without bending them and to maintain the flattening of the other lids being transported by the conveyor. These other lids are located in line with the other suction grooves denoted 56, 60, 62, 64, 62′, 64′, 62″, 64″. Suction in the unloading suction groove is stopped at the time of or immediately after the activation of suction through the gripping members of the removal device.

The at least one slide profile comprises at least one vertical through-passage, the conveyor belt comprising at least one hole adjacent to the orifice, and a removal device for removing at least one lid, said removal device comprising at least one gripping member and a translation-driving device for said at least one gripping member, said gripping member being capable of passing through the at least one passage of the slide profile and the hole of the conveyor belt, when the hole is arranged directly opposite the at least one passage, in order to detach the lid from the conveyor belt.

The hole is positioned between the orifice and the additional orifice, the at least one passage being positioned between the suction groove and the additional suction groove.

Advantageously, this arrangement facilitates separating the lid from the conveyor belt during unloading.

The invention also relates to a transport and decontamination machine which comprises a conveyor according to the features mentioned above and a decontamination device arranged below a portion of the at least one conveyor belt, said decontamination device being able to decontaminate a main face of at least one lid fixed to the conveyor belt.

### BRIEF DESCRIPTION OF FIGURES

FIG. 1 is a side perspective view of a conveyor according to this disclosure;

FIG. 2 is a partial side perspective view of a portion of the conveyor according to this disclosure;

FIG. 3 is another partial side perspective view of a portion of the conveyor according to this disclosure;

FIG. 4 is a perspective view of a portion of the conveyor belt of the conveyor illustrated in FIG. 1;

FIG. 5 is a perspective view from below of a slide profile of the conveyor illustrated in FIG. 1;

FIG. 6A is a perspective view of a first lid storage magazine of the conveyor illustrated in FIG. 1;

FIG. 6B is a perspective view of a second lid storage magazine of the conveyor illustrated in FIG. 1;

FIG. 7 is a perspective view of a destacking device which transfers lids from the magazine to the conveyor in accordance with this disclosure;

FIG. 8 is a side perspective view of a removal device for removing lids from the conveyor to place on the container in accordance with this disclosure;

FIG. 9 is a side perspective view of a portion of the conveyor illustrated in FIG. 1, in a movement position of a conveyor belt;

FIG. 10 is a side perspective view of a portion of the conveyor illustrated in FIG. 1, in a lid removal position;

FIG. 11 is a schematic view of a lid transport and decontamination machine according to this disclosure;

FIG. 12 is a schematic view of a slide profile according to this disclosure; and

FIG. 13 is a perspective view of a portion of a variant conveyor belt of the conveyor.

### DETAILED DESCRIPTION

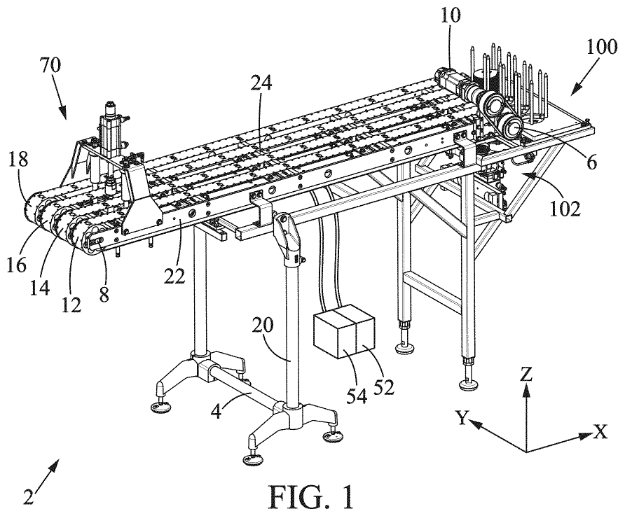

In this patent application, the terms “horizontal”, “vertical”, “top”, and “bottom” are to be interpreted in conjunction with the positions shown in the figures. In this patent application, the term “lid” is used to designate any type of rigid or flexible cover. Referring to FIG. 1, the conveyor 2 according to this disclosure is capable of transporting lids. Conveyor 2 comprises a frame 4, a driving first pulley 6 and a free second pulley 8 which are carried by the frame, a rotation-driving device 10 for the first pulley, and four endless conveyor belts 12, 14, 16, 18 mounted around the first pulley and second pulley.

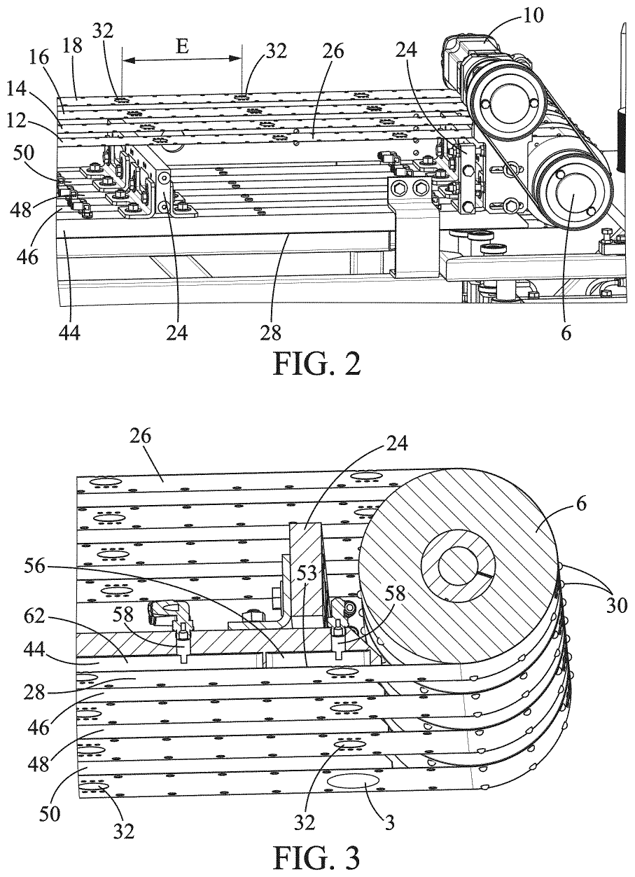

Frame 4 comprises feet 20, two side members 22 carried by the feet, and cross-members 24. Side members 22 extend in a longitudinal direction X. Cross-members 24 are fixed at each of their ends to the side members. The cross-members extend in a transverse direction Y perpendicular to the longitudinal direction X. The cross-members are arranged between an upper horizontal portion 26 of the conveyor belts and a lower horizontal portion 28 of the conveyor belts, as can be seen in FIGS. 2 and 3.

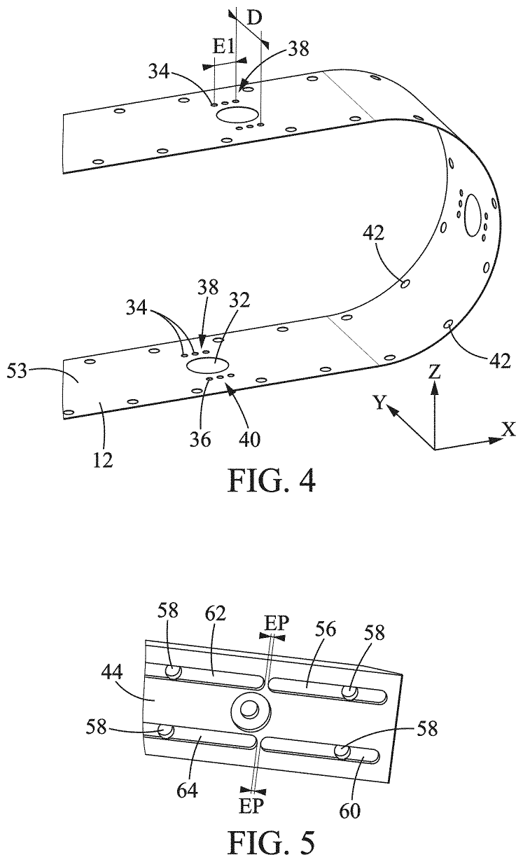

First pulley 6 is mounted so as to rotate about an axle fixed to one end of side members 22 and perpendicular to them. First pulley 6 is located on the side of the conveyor called the “loading side” in the following description. Second pulley 8 is mounted so as to rotate freely about an axle fixed to the other end of the side members 22 and perpendicular to them. Second pulley 8 is located on the side of the conveyor called the “unloading side” in the following description. In the embodiment shown, first pulley 6 and second pulley 8 comprise drive pins 30 suitable for insertion into holes 42 of the belt visible in FIG. 4.

Driving device 10 is, for example, a gearbox motor unit. It is fixed to frame 4. The four conveyor belts 12, 14, 16, 18 extend along the longitudinal direction X between the pulleys, parallel to each other. Referring to FIG. 4, each conveyor belt 12, 14, 16, 18 has through-holes 32, through-orifices 34, and additional through-orifices 36.

Holes 32 are aligned along the longitudinal direction X and centered relative to the longitudinal edges of the conveyor belts. Two adjacent holes 32 are separated by a constant center-to-center distance E along the longitudinal direction X, which corresponds to the removal spacing for the lids. Center-to-center distance E is for example equal to 165 mm. The conveyor belt has a length that is a multiple of center-to-center distance E.

Orifices 34 are aligned along the longitudinal direction X. They are adjacent to the holes. In particular, in the embodiment shown, orifices 34 form a set 38 of several orifices aligned along the longitudinal direction X and grouped next to a hole 32. Each set of orifices 38 is arranged next to a hole. Two adjacent sets of orifices 38 are separated by a constant distance. Center-to-center distance E1 between the orifices farthest apart in a set is greater than the thickness EP of the wall between a first suction groove 56 and a second suction groove 62, as described further below. Additional orifices 36 are similar to orifices 34. They will not be described again in detail. Sets of additional orifices 40 extend along the side of each hole that is opposite to the side provided with sets of orifices 38. Thus, each hole 32 is located between, and centered relative to, a set of orifices 38 and a set of additional orifices 40. The distance D in the transverse direction Y between a set of orifices 38 and a set of additional orifices 40 is less than the diameter of the transported lids. In particular, orifices 34 and additional orifices 36 are inscribed inside the diameter of the smallest lid to be transported in a center-to-center distance E1 along the longitudinal direction X and within a distance D along the transverse direction Y. Preferably, the conveyor belts are made of stainless steel.

In the embodiment shown, the side edges of each conveyor belt 12, 13, 14, 16 comprise holes 42 able to take turns engaging with drive pins 30 of the pulleys during rotation of the pulleys. Holes 42 are arranged at a constant spacing that is a multiple of the center-to-center distance E.

Referring to FIGS. 2, 3, and 5, conveyor 2 comprises four slide profiles 44, 46, 48, 50 carried by the frame 4. Conveyor 2 also comprises a pump 52 and a valve block 54 connected to pump 52. Valve block 54 comprises a supply cut-off system by means of solenoid valves and an electronic control unit for the supply cut-off system. This electronic control unit is for example composed of an ASIC or a processor. The valve block is capable of selectively controlling the opening and closing of the first, second, third, fourth, and fifth solenoid valves so that air is drawn into the suction grooves denoted 56, 60, 62, 64, 62′, 64′, 62″, 64″ and into unloading suction grooves 62′″, 64′″ during a lid loading and/or transport phase and so that air is drawn only into the suction grooves denoted 56, 60, 62, 64, 62′, 64′, 62″, 64″ during a lid unloading phase. Air is not drawn into unloading suction grooves 62′″, 64′″ during lid unloading, as described below.

Pump 52 is a vacuum pump, for example of the Venturi type. The pump and valve block are schematically represented in FIG. 1.

Alternatively, the conveyor comprises several pumps 52, 54.

Slide profiles 44, 46, 48, 50 are fixed below cross-members 24 and are carried by them. They extend along the longitudinal direction X. One of the conveyor belts surrounds each slide profile. Each slide profile is arranged between an upper horizontal portion 26 of a conveyor belt and a lower horizontal portion 28 of a conveyor belt, as shown in FIG. 2.

For better comprehension, a side member 22 has not been shown in FIG. 2. Thus the lower face of slide profiles 44, 46, 48, 50 is facing main faces 53 of conveyor belts 12, 14, 16, 18. The lower inner face of conveyor belts 12, 14, 16 and 18 is in contact with the lower face of slide profiles 44, 46, 48, 50.

Referring to FIG. 5, slide profile 44 comprises a first suction groove 56 extending along the longitudinal direction X. Slide profile 44 further comprises a first additional suction groove 60, at least one second suction groove 62, and at least one second additional suction groove 64. Grooves 60, 62, 64 extend along the longitudinal direction X.

First additional suction groove 60 is arranged parallel to first suction groove 56, at a distance therefrom in the transverse direction Y that is equal to the distance between set of orifices 38 and set of additional orifices 40 in the transverse direction Y. Second suction groove 62 lies in the extension of first suction groove 56. It is aligned with first suction groove 56. Second additional suction groove 64 is positioned in the extension of first additional suction groove 60. It is parallel to second suction groove 62.

For example, in the embodiment shown in FIG. 12, slide profile 44 has a first suction groove 56, a second suction groove 62, a third suction groove 62′, a fourth suction groove 62″, and a fifth suction groove 62″, which are aligned with each other.

This slide profile further comprises a first additional suction groove 60, a second additional suction groove 64, a third additional suction groove 64′, a fourth additional extension groove 64″, and a fifth additional extension groove 64″, which are aligned with each other.

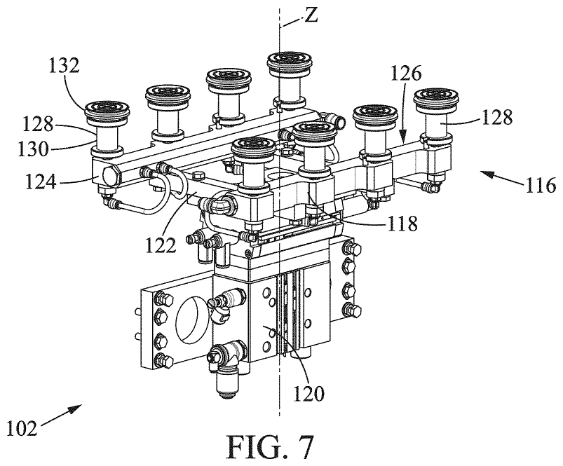

First suction groove 56 and first additional suction groove 60 form a first suction zone. This first suction zone is located above the destacking device illustrated in FIG. 7.

Second suction groove 62, second additional suction groove 64, third suction groove 62′, third additional suction groove 64′, fourth suction groove 62″, and fourth additional suction groove 64″ form a second suction zone. This second suction zone is a lid transport zone.

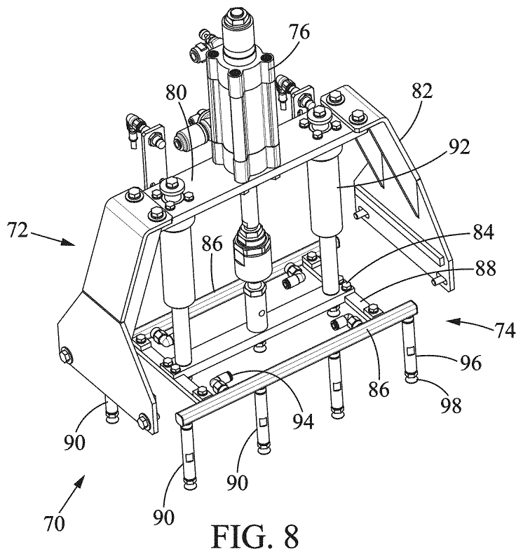

Fifth suction groove 62′″ and fifth additional suction groove 64′″ form a third suction zone located at the lid removal device illustrated in FIG. 8. To facilitate understanding the invention, fifth suction groove 62′″ and fifth additional suction groove 64′″ are also called unloading suction grooves.

The open sides of suction grooves 56, 60, 62, 62′, 62″, 62′″, 64, 64′, 64″, 64′″ of slide profiles 44 to 50 are directly facing main face 53 of the conveyor belts. The suction grooves denoted 56, 62, 62′, 62″, 62′″ are positioned so that sets of orifices 38 of the conveyor belts take turns being in line with the suction grooves denoted 56, 62, 62′, 62″, 62″, during movement of the conveyor belts. Suction grooves denoted 60, 64, 64′, 64″, 64′″ are positioned so that sets of additional orifices 40 of the conveyor belts take turns being in line with suction grooves denoted 60, 64, 64, 64′, 64″, 64″ during movement of the conveyor belts. The existence of two sets of parallel suction grooves 56, 60 makes it possible to increase the suction force applied to a lid 3 during its transport by a conveyor belt.

The thickness EP of the wall between first suction groove 56 and second suction groove 62 is less than the center-to-center distance E1 between the orifices farthest apart within a set of orifices 38 of the conveyor belt. Similarly, the thickness EP of the wall between first additional suction groove 60 and first additional suction groove 64 is less than the center-to-center distance between the orifices farthest apart within a set of additional orifices 40 of the conveyor belt. This thickness Ep exists between all suction grooves aligned in the longitudinal direction.

Slide profiles 48 to 50 are identical to slide profile 44.

The bottom wall of each suction groove 56, 60, 62, 64 has an opening 58 connected to valve block 54. Openings 58 of suction grooves 56 are connected to valve block 54 by hoses so that air is drawn in through the suction grooves. Second suction grooves 62 of the slide profiles are connected to valve block 54. Suction through first suction grooves 56 can be controlled independently of the suction through second suction grooves 62, by means of solenoid valves. It is possible to stop the pumping into first suction grooves 62 while maintaining the pumping into second suction grooves 56, for example when loading lids located above the first suction grooves. It is thus possible to stop pumping into first suction grooves 62 while continuing to pump into second suction grooves 56, for example when loading lids positioned opposite the first suction grooves. It is thus possible to engage or lift the suction depending on the cycle of the conveyor, for example during a lid destacking cycle, during a lid removal cycle, or during a lid loading cycle.

Preferably, each assembly of suction groove and additional suction groove is supplied by a same suction source, in order to limit energy consumption of the suction pumps during startup.

Thus, with the slide profile illustrated in FIG. 12, first suction grooves 56 and 60 are connected to the same solenoid valve of the valve block, and second suction grooves 62 and 64 are connected to another solenoid valve of the valve block. Third suction grooves 62′ and 64′ are connected to a third solenoid valve. Fourth suction grooves 62″ and 64″ are connected to a fourth solenoid valve. Finally, fifth suction grooves 62″ and 64″ are connected to a fifth solenoid valve. When conveyor 2 has a large dimension in the longitudinal direction X, each slide profile may comprise several suction grooves 62, one lying in the extension of the other, and possibly several additional suction grooves 64 each lying in the extension of the other.

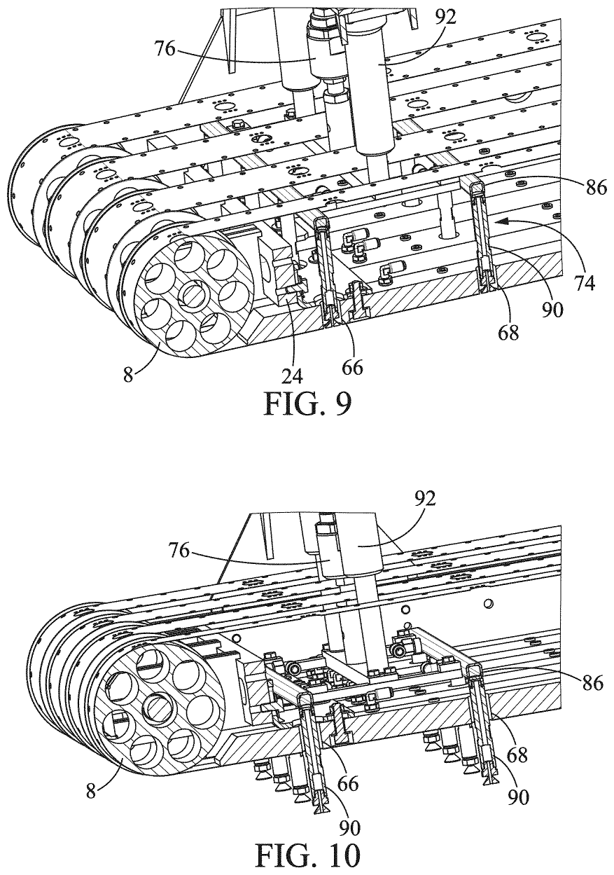

Referring to FIGS. 9 and 10, each slide profile 44, 46, 48, 50 further comprises two vertical through-passages 66, 68 located on the unloading side of the conveyor. Passages 66, 68 are positioned between second suction groove 62 and second additional suction groove 64. In particular, passages 66 are centered along the transverse direction Y. The diameter of passages 66, 68 is substantially equal to the diameter of holes 32 of the conveyor belts. Passages 66, 68 are positioned on the slide profiles so that holes 32 of conveyor belts 12, 14, 16, 18 take turns being in line with passages 66, 68, during movement of the conveyor belt. In particular, the center-to-center distance between the two passages 66, 68 is equal to the center-to-center distance E between two consecutive holes 32 of the conveyor belts.

Referring to FIGS. 1 and 8, the conveyor further comprises a lid removal device 70 capable of unloading the lids in contact with the conveyor belts and depositing them on supports such as containers. Removal device 70 is fixed to frame 4 on the unloading side of the conveyor.

Referring to FIG. 8, removal device 70 comprises a support 72, a gripping assembly 74 movable in vertical translation relative to the support, and a vertical-translation-driving device 76 capable of driving the movement of the gripping assembly relative to the support.

Support 72 comprises a transverse plate 80 and two support arms 82 each fixed to one end of the transverse plate and to side members 22. Support arms 82 are capable of carrying transverse plate 80 above the conveyor.

Gripping assembly 74 is carried by support 72. It comprises a central slat 84, two transverse pipes 86 fixed to the central slat by bars 88, and eight gripping members 90 fixed to pipes 86. Pipes 86 are apart from each other by a distance equal to the distance between two passages 66, 68 of a same slide profile. Central slat 84 is fixed to transverse plate 80 of the support by two column/socket guide devices. Vertical-translation-driving device 76 is fixed to transverse plate 80. It is capable of moving gripping assembly 74 relative to support 72, between a rest position and a position for unloading lids from the conveyor belts.

Pipes 86 comprise connectors 94 connected to either a new solenoid valve of valve block 54 or to a new pump.

Each gripping member 90 comprises a channel portion 96 having one end in communication with a pipe 86 and an opposite end provided with a suction cup 98. Suction cups 98 as well as the suction force of the air coming from valve block 54 via a pipe make it possible to retain lids 3 against gripping members 90 after they have been removed from the conveyor belts.

Gripping members 90 are separated from each other in the longitudinal direction X by a distance equal to the center-to-center distance E which corresponds to the distance between passages 66, 68 of two adjacent slide profiles 44 to 50. Gripping members 90 are separated from each other in the transverse direction Y by the same center-to-center distance as the center-to-center distance between slide profiles 44 to 50. The dimension of gripping members 90 is less than the dimension of passages 66, 68 of the slide profiles.

In the rest position illustrated in FIG. 9, gripping assembly 74 is arranged between an upper horizontal portion 26 of the conveyor belts and a lower horizontal portion 28 of the conveyor belts. The end of gripping members 90 is arranged in passages 66, 68 of the slide profiles.

In the unloading position, illustrated in FIG. 10, holes 32 of conveyor belts 12, 14, 16, 18 are facing passages 66, 68, and driving device 76 moves gripping assembly 74 downwards in order to remove lids 3 from conveyor belts 12, 14, 16, 18 and place them on another support such as containers.

The stroke of the vertical displacement of gripping members 90 is less than the distance between the upper horizontal portion of the conveyor belt and the lower horizontal portion of the conveyor belt.

Referring to FIG. 1, the conveyor 2 further comprises at least one lid storage magazine 100 and a destacking device 102 able to transfer lids from the lid storage magazine to the conveyor. Lid storage magazine 100 is fixed to frame 4 on the loading side, in the extension of slide profiles 44 to 50 along the longitudinal direction X.

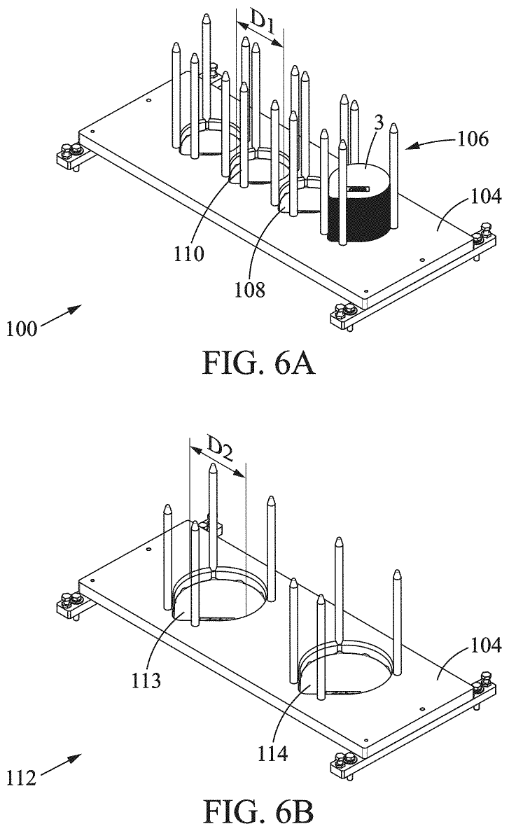

Referring to FIGS. 6A and 6B, lid storage magazine 100 comprises a plate 104 provided with a row of loaders 106. In the embodiment shown, plate 104 comprises four loaders. Each loader 106 is arranged in alignment with a conveyor belt. Each loader 106 comprises a dispensing opening 108 formed in the plate and a guide 110 fixed around each dispensing opening in order to keep the stack of lids 3 together. Screw heads, not shown, are fixed to the vertical edges of each dispensing opening 108 in order to retain by the edges the lids 3 stacked on the opening. In the embodiment shown by way of example, guides 110 each consist of four vertical rods distributed and fixed around the dispensing openings.

Advantageously, conveyor 2 comprises at least two lid storage magazines 100, 112 removably fixed to frame 4. Lid storage magazines 100, 112 are each capable of storing lids of different sizes. They are interchangeable according to the size of the lids to be transported. Thus, conveyor 2 may comprise a first lid storage magazine 100 having dispensing openings of a first diameter D1 and a second lid storage magazine 112 having dispensing openings of a second diameter D2 that is different than the first diameter D1. For example, the first magazine is capable of storing lids 3 with a maximum diameter of 95 millimeters, and the second magazine is capable of storing lids having a diameter of between 96 and 150 millimeters. When the diameter of lids 3 is much greater than the width of a conveyor belt, second lid storage magazine 112 may comprise only two loaders 106 and therefore only two dispensing openings 108.

Dispensing openings 108 are then arranged in the extension either of the first belt and third belt, or of the second belt and fourth belt.

In this case, in general, if the conveyor has a number n of conveyor belts, first storage magazine 100 comprises a number n of dispensing openings and second storage magazine 112 comprises a number n/2 of dispensing openings.

Destacking device 102 is positioned below the storage magazine and below an end portion of slide profiles 44 to 50 on the loading side. Referring to FIG. 7, destacking device 102 comprises a gripping assembly 116, a rotation-driving device 118, and a vertical-translation-driving device 120.

Gripping assembly 116 comprises a framework 122, two transverse pipes 124, 126 fixed to the framework and parallel to one another, and gripping members 128 fixed to a pipe and in fluid communication therewith. The number of gripping members mounted on each pipe is preferably equal to the number of loaders of the lid storage magazine fixed to the framework. In the embodiment shown, four gripping members 128 are fixed to first pipe 124 and are in communication therewith. The four gripping members are aligned along a first row. Four gripping members are fixed to second pipe 126 and are in communication therewith. The four gripping members are aligned along a second row.

Each gripping member 128 comprises a vertical hollow channel 130 having an open upper end and a lower end connected to a pump by a hose.

First pipe 124 is connected to the fourth pump and second pipe 126 is connected to the second pump, such that suction can be implemented independently for the first row of gripping members and for the second row of gripping members.

Advantageously, gripping members 128 may comprise suction cups 132 capable of being fixed to the upper end of channels 130. Advantageously, suction cups of different sizes can be mounted on the upper end of the channels, the size of the suction cups being chosen according to the size of the lids.

Gripping assembly 116 is mounted so as to rotate about a vertical axis Z-Z centered relative to the first and second pipes. The vertical axis Z-Z is located between and equidistant from the first and second pipes. The vertical axis Z-Z is also arranged between the end of the slide profiles and the lid storage magazine.

Rotation-driving device 118 is capable of causing the gripping assembly to pivot about the vertical axis Z-Z by half a turn so that the gripping members of the first row and second row take turns being below the openings of the lid storage magazine on the one hand and below sets of orifices 38, 40 of the conveyor belts on the other hand.

The gripping assembly is also mounted so as to move vertically in translation. The translation-driving device 120 is capable of moving gripping assembly 74 vertically between a high position for picking up/removing lids and a low position for rotating the lids. The rotation- and translation-driving devices are, for example, electric actuators, pneumatic or hydraulic cylinders.

Alternatively, the conveyor comprises a number n of conveyor belt(s) and a number n of slide profile(s), the number n being different than four. For example, the number n may be equal to one, two, or three, or be greater than four. The number of pumps or of suction areas may be different. For example, two different pumps can be connected, one to the suction groove and the other to the additional suction groove. Alternatively, the conveyor also comprises a rotation-driving device for the second pulley.

Alternatively, the first and second pulleys may be replaced by an assembly of several pulleys which can be rotated independently of each other, in order to be able to reduce the number of lids to be transported if needed.

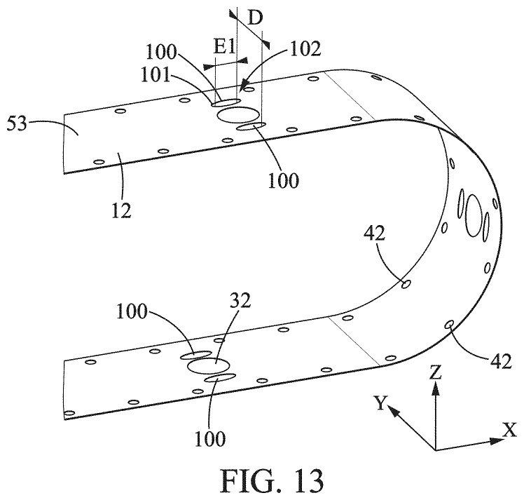

According to an alternative illustrated in FIG. 13, each set of orifices and each set of additional orifices is replaced by an oblong-shaped orifice 100 extending along the longitudinal direction X. Oblong-shaped orifices 100 are arranged one on either side of each hole 32. They are aligned along the longitudinal direction. The oblong orifice has a first end 101 and an opposite second end 102 along the longitudinal direction X. The center-to-center distance E1 between first end 101 and second end 102 is greater than the thickness EP of the wall between first suction groove 56 and second suction groove 62.

Alternatively, slide profiles 44 to 50 comprise a single passage 66 and the removal device 70 comprises only one pipe 86.

According to a less advantageous alternative, the grooves of the slide profiles are replaced by pipes extending longitudinally, the pipes comprising several aligned openings which open onto an outer face of the slide profiles.

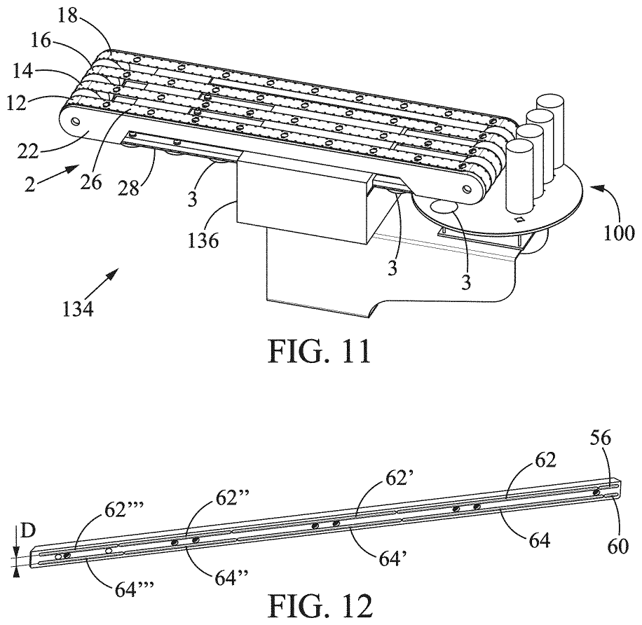

With reference to FIG. 11, the invention also relates to a transport and decontamination machine 134 which comprises a conveyor 2 as described above, and a decontamination device 136 arranged below or around the lower horizontal portion 28 of the conveyor belts. The decontamination device is capable of decontaminating a main face of the lids, this main face being opposite to main face 53 which is directly facing a slide profile. The decontamination device is for example an ultraviolet decontamination device, a pulsed light decontamination device, or a peroxide decontamination device.

During operation, loaders 106 of the lid storage magazine comprise lids 3 stacked on top of each other. Gripping members 128 of destacking device 102 are moved in translation to a high position for picking up/removing lids. Gripping members 128 of the first row are arranged under the dispensing openings 108. The pump connected to the destacking device sucks down the lids stored in the loaders. Gripping members 128 of the first and second rows are moved vertically to a low position. They rotate half a turn, then are moved vertically to a high position. Gripping members 128 of the first row are then pressed against conveyor belts 12,14,16,18. The pump connected to the gripping members of the first row is stopped. Suction is generated through first suction groove 56 and first additional suction groove 60. Lids 3 are transferred by suction from destacking device 102 to conveyor belts 12, 14, 16, 18 by means of the suction coming from the suction grooves and passing through sets of orifices 38, 40. When lids 3 are fixed by suction to the conveyor belts, the conveyor belts and lids 3 move along the longitudinal direction X towards the unloading side. The portions of the conveyor belts that are carrying lids are then located opposite second suction grooves 62 and second additional suction grooves 64. Next they are transported from one suction groove to another, all the way to the removal area corresponding to fifth suction grooves 62′″ and 64″. When the lids reach alignment with passage 66 or 68, suction is stopped through fifth suction grooves 62′″ and fifth additional suction grooves 64″. Suction through gripping members 90 of removal device 70 is engaged. Suction cups 98 take up the lids. Suction is maintained through suction grooves denoted 62, 64, 62′, 64′, 62″, 64″, 62′″, 64′″ which correspond to a lid transfer and decontamination zone. Gripping members 90 are moved vertically downward as illustrated in FIG. 10. Gripping members 90 place the lids on a support, such as containers for example.

According to an alternative not shown, the slide profile comprises only a first suction groove extending in the longitudinal direction and a second suction groove aligned with and in the extension of the first suction groove. The removal device is arranged in line with the second suction groove, and the valve block is capable of controlling the pump to draw air into the first suction groove and into the second suction groove during the conveyor belt loading phase and/or the lid transport phase. And the valve block is capable of controlling the pump to draw air only into the first suction groove during the lid unloading phase by the removal device. No air is drawn through the second suction groove during the lid unloading phase. The second suction groove constitutes an unloading suction groove.

1-15. (canceled)

16. A lid transport conveyor comprising:
a frame,
at least a first pulley and a second pulley which are carried by the frame,
a rotation-driving device capable of rotating at least the first pulley,
at least one endless conveyor belt mounted between the first pulley and the second pulley, the conveyor belt extending in a longitudinal direction, the conveyor belt being driven to move by the first pulley,
at least one slide profile carried by the frame, the slide profile comprising a first suction groove extending in the longitudinal direction, the open side of the first suction groove directly facing a main face of the conveyor belt, and
at least one pump capable of drawing air through said first suction groove,
said conveyor belt comprising at least one through-orifice positioned, at least at times, in line with the first suction groove, during movement of the conveyor belt; at least one lid being held by suction in contact with a face of the conveyor belt that is opposite to the main face, when the orifice is directly opposite the first suction groove,
wherein the slide profile comprises at least one second suction groove aligned with and in the extension of the first suction groove, the open side of the second suction groove directly facing a main face of the conveyor belt, said at least one pump being capable of drawing air through said second suction groove.

17. The conveyor according to claim 16, wherein the at least one conveyor belt comprises at least one set of several orifices grouped together and aligned along the longitudinal direction, the slide profile comprising a wall between the first suction groove and the second suction groove, and wherein the center-to-center distance between the orifices farthest apart within a set of orifices is greater than the thickness of the wall between the first suction groove and the second suction groove.

18. The conveyor according to claim 16, wherein the at least one conveyor belt comprises at least one oblong orifice extending in the longitudinal direction, the oblong orifice having a first end and an opposite second end along the longitudinal direction; the slide profile comprising a wall between the first suction groove and the second suction groove and wherein the center-to-center distance between the first and second ends of the oblong orifice is greater than the thickness of the wall between the first suction groove and the second suction groove.

19. The conveyor according to claim 16, wherein the slide profile comprises at least one additional first suction groove parallel to the first suction groove, and wherein the conveyor belt has at least one additional orifice, the at least one additional orifice of the conveyor belt being arranged, at least at times, in line with said first additional suction groove, during movement of the conveyor belt.

20. The conveyor according to claim 16, comprising several slide profiles and several conveyor belts extending in the longitudinal direction, the slide profiles being parallel to each other and arranged next to each other in a transverse direction, the conveyor belts being parallel to each other and arranged next to each other in a transverse direction.

21. The conveyor according to claim 16, wherein the conveyor belt is made of stainless steel.

22. The conveyor according to claim 16, wherein the at least one slide profile is arranged between an upper horizontal portion of the at least one conveyor belt and a lower horizontal portion of the at least one conveyor belt.

23. The conveyor according to claim 16, further comprising a lid storage magazine among a first lid storage magazine and a second lid storage magazine, the first lid storage magazine and the second lid storage magazine being suitable for mounting on the frame in a removable manner, the first lid storage magazine including a plate comprising dispensing openings having a first diameter, the second lid storage magazine including a plate comprising dispensing openings having a second diameter that is different from the first diameter.

24. The conveyor according to claim 23, comprising N slide profiles and N conveyor belts, the number N being a positive integer greater than 1, and wherein the plate of the first lid storage magazine comprises N dispensing openings, the plate of the second lid storage magazine comprising N/2 dispensing openings.

25. The conveyor according to claim 16, comprising a lid destacking device capable of transferring the lids from the lid storage magazine to the at least one conveyor belt, the destacking device comprising:
at least two gripping members rotatably mounted relative to a vertical axis arranged between the two gripping members,
a rotation-driving device capable of rotating the gripping members about the vertical axis;
the gripping members being mounted to be vertically movable in translation,
a translation-driving device capable of driving the gripping members in translation between a high position for picking up/removing lids and a low position for transferring lids.

26. The conveyor according to claim 25, wherein the lid storage magazine is fixed to the frame in the extension of the slide profiles along the longitudinal direction, and wherein the destacking device is positioned below the lid storage magazine and below one end of the slide profiles, the vertical axis being arranged between one end of the slide profiles and the lid storage magazine.

27. The conveyor according to claim 16, wherein the slide profile comprises at least one unloading suction groove, aligned with the first suction groove, the open side of the unloading suction groove directly facing a main face of the conveyor belt, said at least one pump being capable of drawing air through said unloading suction groove; the conveyor comprising a removal device for removing at least one lid arranged in line with an unloading suction groove, and a valve block capable of controlling the at least one pump to draw air into the at least one first suction groove and into the unloading suction groove during a lid transport phase and/or lid loading phase, and to draw air only into the unloading suction groove during a phase of unloading at least one lid by the removal device.

28. The conveyor according to claim 27, wherein the at least one slide profile comprises at least one vertical through-passage, the conveyor belt comprising at least one hole adjacent to the orifice, and wherein said removal device comprises at least one gripping member and a vertical translation-driving device for said at least one gripping member, said gripping member being capable of passing through the at least one passage of the slide profile and the hole of the conveyor belt, when the hole is arranged directly opposite the at least one passage, in order to detach the lid from the conveyor belt.

29. The conveyor according to claim 28, wherein the slide profile comprises at least one additional first suction groove parallel to the first suction groove, and wherein the conveyor belt comprises at least one additional orifice, the at least one additional orifice of the conveyor belt being arranged, at least at times, in line with said first additional suction groove, during movement of the conveyor belt, and wherein the hole is positioned between the orifice and the additional orifice, the at least one passage being positioned between the first suction groove and the first additional suction groove.

30. A transport and decontamination machine comprising:
a) a lid transport conveyor comprising:
a frame,
at least a first pulley and a second pulley which are carried by the frame,
a rotation-driving device capable of rotating at least the first pulley,
at least one endless conveyor belt mounted between the first pulley and the second pulley, the conveyor belt extending in a longitudinal direction, the conveyor belt being driven to move by the first pulley,
at least one slide profile carried by the frame, the slide profile comprising a first suction groove extending in the longitudinal direction, the open side of the first suction groove directly facing a main face of the conveyor belt,
at least one pump capable of drawing air through said first suction groove,
said conveyor belt comprising at least one through-orifice positioned, at least at times, in line with the first suction groove, during movement of the conveyor belt; at least one lid being held by suction in contact with a face of the conveyor belt that is opposite to the main face, when the orifice is directly opposite the first suction groove,
wherein the slide profile comprises at least one second suction groove aligned with and in the extension of the first suction groove, the open side of the second suction groove directly facing a main face of the conveyor belt, said at least one pump being capable of drawing air through said second suction groove, and
b) a decontamination device arranged below a portion of the at least one conveyor belt, said decontamination device being able to decontaminate a main face of at least one lid fixed to the conveyor belt.
